(19) United States
(12) Patent Application Publication
(10) Pub. No.: US 2024/0226303 A9
(43) Pub. Date: Jul. 11, 2024

(54) NOVEL COMPOSITION

(71) Applicant: RECKITT BENCKISER HEALTH LIMITED, Slough, Berkshire (GB)

(72) Inventors: Ammar ALMAJAAN, Loughborough, Leicestershire (GB); Gavin Paul ANDREWS, Belfast (GB); David Simon JONES, Belfast (GB); Mariana Palmeira BEZERRA, Leicester (GB); Walkiria Santos SCHLINDWEIN, Leicester (GB); Karina WOJDAT, Leicester (GB)

(21) Appl. No.: 18/548,645

(22) PCT Filed: Mar. 4, 2022
(86) PCT No.: PCT/GB22/50579

§ 371 Date: Sep. 1, 2023

(30) Foreign Application Priority Data
Mar. 4, 2021 (GB) 2103073.9
Jul. 9, 2021 (GB) 2109929.6
Aug. 27, 2021 (GB) 2112322.9

(51) Int. Cl. A61K 47/32; A61K 31/167; A61K 31/192; A61K 31/196; A61K 31/405; A61K 31/5415; A61K 31/616

(52) U.S. Cl. CPC A61K 47/32; A61K 31/167; A61K 31/192; A61K 31/196; A61K 31/405; A61K 31/5415; A61K 31/616

(57) ABSTRACT

A solidified melt extrudate comprising an active pharmaceutical ingredient in an amorphous form and a combination of at least two polymers.

The present invention is directed to a novel pharmaceutically active ingredient-containing composition. In particular, the present invention is directed to an NSAID-containing composition in which the NSAID is in an amorphous form. A preferred NSAID is amorphous ibuprofen.

It is known that the crystalline form of an active pharmaceutical ingredient is a more stable form than the amorphous form of the same compound. As a result, the crystalline form is less soluble. However, as amorphous forms are typically not as physically stable as the corresponding crystalline form they readily convert into the corresponding crystalline form. Accordingly, while crystalline forms have high stability but low solubility, amorphous forms have high solubility but low stability.

There have been attempts to prepare medicaments containing the active principles in amorphous form. However, the lack of stability has heretofore prevented their use as the active substance in a pharmaceutical composition as the required dissolution profile will not necessarily be consistent or fast.

Accordingly, the crystalline form of an active pharmaceutical ingredient is used in commercial products. However, the use of these materials often requires the inclusion of dissolution aids in the formulation which adds to the cost and complexity of the product.

It would, therefore, be desirable to develop a composition which incorporates an amorphous form of a drug having the required stability.

Polymers are an important component of amorphous solid drug delivery systems. Mainly because they are the determinant of the physical stability as well as the drug release behaviour from the drug delivery system.

Physical stability of an amorphous drug in a polymeric carrier depends on factors such as intermolecular interactions, polymer glass transition temperature (Tg) and the equilibrium solubility of the drug candidate in the corresponding polymer. While the capability of the polymer to improve solid-state stability can be associated with its hydrophobicity, solubility enhancement of a hydrophobic drug can be attained by using hydrophilic polymers.

Therefore, when designing and developing amorphous drug delivery systems a significant challenge lies in finding a polymer or combination of polymers that possesses this dual effect.

The polymer or polymer combination should exhibit acceptable miscibility with the active pharmaceutical ingredient and be able to inhibit precipitation of the active pharmaceutical ingredient from a supersaturated solution.

According to the first aspect of the present invention there is provided a solidified melt extrudate comprising an active pharmaceutical ingredient in an amorphous form and a combination of at least two polymers.

The active pharmaceutical ingredient can be selected from NSAIDs and paracetamol.

The NSAID can be selected from ibuprofen, flurbiprofen, ketoprofen, diclofenac, naproxen, aspirin, indomethacin, and meloxicam. A preferred NSAID is ibuprofen.

The extrudate can comprise up to 50% by weight of the active pharmaceutical ingredient.

The extrudate can comprise up to 45% by weight of the active pharmaceutical ingredient.

The extrudate can comprise up to 40% by weight of the active pharmaceutical ingredient.

The extrudate can comprise at least 10% by weight of the active pharmaceutical ingredient.

The extrudate can comprise at least 15% by weight of the active pharmaceutical ingredient.

The extrudate can comprise at least 20% by weight of the active pharmaceutical ingredient.

The extrudate can comprise at least 25% by weight of the active pharmaceutical ingredient.

The extrudate can comprise at least 30% by weight of the active pharmaceutical ingredient.

The extrudate can comprise from 10% by weight to 50% by weight of the active pharmaceutical ingredient. The extrudate can comprise from 15% by weight to 45% by weight of the active pharmaceutical ingredient. The extrudate can comprise from 20% by weight to 40% by weight of the active pharmaceutical ingredient. The extrudate can comprise from 25% by weight to 40% by weight of the active pharmaceutical ingredient. The extrudate can comprise from 30% by weight to 40% NSAID by weight of the active pharmaceutical ingredient.

Typically, the combination of polymers is miscible with the active pharmaceutical ingredient.

The first polymer of the combination of at least two polymers can be selected from polymers having a glass transition temperature of at least 30° C. The polymer can have a glass transition temperature of at least 40° C. The polymer can have a glass transition temperature of less than or equal to 60° C. The polymer can have a glass transition temperature of between 40° C. and 50° C.

The first polymer is selected such that it provides stability for the amorphous form of the active pharmaceutical ingredient for a period of at least six months.

Typically, the first polymer of the combination of two polymers can be selected from polymethacrylates including but not limited to amino methacrylate copolymer, amino-methyl methacrylate, methacrylic acid methyl methacrylate, hydroxypropyl methylcellulose, ethyl cellulose, hydroxypropyl methylcellulose acetate succinate, polyethylene glycol, poly(vinylmethyl ether/maleic anhydride), crospovidone, croscarmellose sodium, dimethylaminoethyl methacrylate co-polymer. A preferred polymer is dimethylaminoethyl methacrylate co-polymer.

Alternatively, the first polymer can be selected from a combination of polymers with the proviso that the combination is selected such that it provides stability for the amorphous form of the active pharmaceutical ingredient for a period of at least six months. The first polymer can be a combination of polymers selected from polymethacrylates including but not limited to amino methacrylate copolymer, amino-methyl methacrylate, methacrylic acid methyl methacrylate, hydroxypropyl methylcellulose, ethyl cellulose, hydroxypropyl methylcellulose acetate succinate, polyethylene glycol, poly(vinylmethyl ether/maleic anhydride), crospovidone, croscarmellose sodium, dimethylaminoethyl methacrylate co-polymer.

The first polymer of the combination of two polymers can be present at a level 30-90% by weight of the composition. Preferably the first polymer of the combination of two polymers can be present at a level 40-80% by weight of the composition. More preferably, the first polymer of the combination of two polymers can be present at a level 50-70% by weight of the composition. Most the first polymer of the combination of two polymers can be present at a level of about 60% by weight of the composition.

The second polymer of the combination of two polymers can be selected from polymers having a glass transition temperature of at least 80° C. The polymer can have a glass transition temperature of at least 90° C. The polymer can have a glass transition temperature of less than 120° C. The polymer can have a glass transition temperature of between 90° C. and 110° C.

Typically, the second polymer of the combination of two polymers can be selected from polyvinylpyrrolidone, polyvinylpyrrolidone-vinyl acetate co-polymer and polyvinyl caprolactam-polyvinyl acetate-polyethylene glycol graft copolymer. Preferably the second polymer is polyvinylpyrrolidone K12 or polyvinylpyrrolidone-vinyl acetate co-polymer (PVPVA64).

Alternatively, the second polymer can be selected from a combination of polymers with the proviso that the combination is selected such that the extrudate has a tensile strength of at least 3 N/mm². The second polymer can be a combination of polymers selected from polyvinylpyrrolidone, polyvinylpyrrolidone-vinyl acetate co-polymer and polyvinyl caprolactam-polyvinyl acetate-polyethylene glycol graft copolymer.

The second polymer is selected such that the extrudate has a tensile strength of 5-50 N/mm².

The second polymer of the combination of two polymers can be present in a level of 1-30% by weight of the composition. Preferably, the second polymer of the combination of two polymers can be present in a level of 10-25% by weight of the composition. More preferably, the second polymer of the combination of two polymers can be present in a level of 15-25% by weight of the composition. Most preferably, the second polymer of the combination of two polymers can be present in a level of about 20% by weight of the composition.

A preferred combination of the first and second polymers is dimethylaminoethyl methacrylate co-polymer and polyvinylpyrrolidone-vinyl acetate co-polymer (PVPVA64). An alternative preferred combination of the first and second polymers is dimethylaminoethyl methacrylate co-polymer and polyvinylpyrrolidone K12.

The ratio of the first polymer and the second polymer of the combination of two polymers can be selected to be from 10:1 to 1:10. A preferred ratio is from 4:1 to 1:4. A more preferred ratio is from 1:1 to 1:3.

The ratio of the of the active pharmaceutical ingredient, the first polymer and the second polymer can be from 1:1:1 to 1:4:4. Preferably, the ratio of the active pharmaceutical ingredient, the first polymer and the second polymer can be from 1:1.5:1.5 to 1:2.5:2.5. Alternatively, the ratio of the active pharmaceutical ingredient, the first polymer and the second polymer can be from 1:2:1 to 1:4:1.

The active pharmaceutical ingredient is portioned between the each of the first and second polymer of the melt extrudate. Preferably, at least 20% of the of the active pharmaceutical ingredient is in the first polymer. More preferably at least 25% is in the first polymer. Most preferably at least 30% is in the first polymer. Preferably, less than or equal to 50% of the active pharmaceutical ingredient is in the first polymer. More preferably less than or equal to 40% is in the first polymer. Most preferably less than or equal to 35% is in the first polymer.

Preferably, from 20% by weight to 50% by weight of the active pharmaceutical ingredient is in the first polymer of the melt extrudate. More preferably, from 25% by weight to 40% by weight of the active pharmaceutical ingredient is in the first polymer of the melt extrudate. Most preferably, from 30% by weight to 35% by weight of the active pharmaceutical ingredient is in the first polymer of the melt extrudate.

One or more processing aids such as silicon dioxide, talc, magnesium silicate and glyceryl monostearate can be added prior to melt extrusion.

One or more additional processing aids can be added post-melt extrusion selected from silicon dioxide, microcrystalline cellulose, crospovidone, carrageenan, chitosan, pectinic acid, glycerides, beta-cyclodextrin and cellulose derivatives.

The combination of polymers is soluble at a pH of about 1 to about 5. Preferably, the combination of polymers is soluble at a pH of about 2 to about 4. Alternatively, the combination of polymers is soluble at a pH of about 1 to about 3.

Preferably the difference in Hansen solubility parameter between active pharmaceutical ingredient and the combination of the first polymer and the second polymer is less than 10 MPa^{1/2}. More preferably the difference in Hansen solubility parameter between active pharmaceutical ingredient and the combination of the first polymer and the second polymer is less than 7 MPa^{1/2}.

The extrudate is typically prepared using a hot melt extrusion process.

The melt extrusion process is preferably conducted at a temperature of between 80° C. and 110° C. More preferably, the temperature of the extrusion process is between 90° C. and 100° C. Most preferably, the temperature of the extrusion process is between 93° C. and 98° C.

The screw speed of the extruder can be selected from 1 revolution per minute (rpm) to 30 revolutions per minute (rpm). Preferably the screw speed of the extruder is from 5-20 rpm. More preferably the screw speed of the extruder is about 10 rpm.

The melt extrusion process can be carried out at a preferred temperature from 80° C. to 110° C. and at a preferred extruder screw speed from 1 rpm to 30 rpm. More preferably, the melt extrusion temperature can be between 90° C. and 100° C. and the extruder screw speed can be from 5 rpm to 20 rpm. Most preferably, the melt extrusion temperature can be between 93° C. and 98° C. and the extruder screw speed can be about 10 rpm.

Alternatively, the screw speed of the extruder can be selected from 100 revolutions per minute (rpm) to 300 revolutions per minute (rpm). Preferably the screw speed of the extruder is from 150-250 rpm. Preferably the screw speed of the extruder is from 180-220 rpm. Most preferably the screw speed of the extruder is about 200 rpm.

The melt extrusion process can be carried out at a preferred temperature from 80° C. to 110° C. and at a preferred extruder screw speed from 100 rpm to 300 rpm. More preferably, the melt extrusion temperature can be between 90° C. and 100° C. and the extruder screw speed can be from 150 rpm to 200 rpm. Most preferably, the melt extrusion temperature can be between 93° C. and 98° C. and the extruder screw speed can be about 180-220 rpm. Most preferably the melt extrusion temperature can be about 95° C. and the extruder screw speed can be about 200 rpm.

Alternatively, the melt extrusion process can be carried out at a temperature from 115° C. to 125° C. and the screw speed of the extruder can be selected from 100 revolutions per minute (rpm) to 300 revolutions per minute (rpm). Preferably the melt extrusion process can be carried out at a temperature of about 120° C. and the screw speed of the extruder can be selected to be about 200 rpm.

Preferably the extrusion processing conditions are selected such that the torque is less than or equal to 35 Nm. More preferably the extrusion conditions are selected such that the torque is less than or equal to 25 Nm. Most preferably the extrusion conditions are selected such that the torque is less than or equal to 20 Nm.

Preferably the extrusion conditions are selected such that the torque is more than or equal to 5 Nm. More preferably the extrusion conditions are selected such that the torque is more than or equal to 10 Nm. Most preferably the extrusion conditions are selected such that the torque is more than or equal to 15 Nm.

Preferably the extrusion conditions are selected such that the torque is less than or equal to 35 Nm and more than or equal to 5 Nm. More preferably the extrusion conditions are selected such that the torque is less than or equal to 25 Nm and more than or equal to 10 Nm. Most preferably the extrusion conditions are selected such that the torque is less than or equal to 20 Nm and more than or equal to 15 Nm.

Preferably, the solidified melt extrudate comprises ibuprofen in an amorphous form and a combination of dimethylaminoethyl methacrylate co-polymer and polyvinylpyrrolidone-vinyl acetate co-polymer (PVPVA64) wherein the extrudate can comprise from 30%-40% by weight ibuprofen wherein the extrudate can be prepared using a hot melt extrusion process at a temperature between 93° C. and 98° C. and wherein the extruder screw speed can be about 10 rpm.

According to a second aspect of the present invention there is provided a solidified melt extrudate consisting essentially of an active pharmaceutical ingredient in an amorphous form, a combination of at least two polymers.

The active pharmaceutical ingredient can be selected from NSAIDs and paracetamol.

The NSAID can be selected from ibuprofen, flurbiprofen, ketoprofen, diclofenac, naproxen, aspirin, indomethacin, and meloxicam. A preferred NSAID is ibuprofen.

The extrudate can comprise up to 50% by weight of the active pharmaceutical ingredient.

The extrudate can comprise up to 45% by weight of the active pharmaceutical ingredient.

The extrudate can comprise up to 40% by weight of the active pharmaceutical ingredient.

The extrudate can comprise at least 10% by weight of the active pharmaceutical ingredient.

The extrudate can comprise at least 15% by weight of the active pharmaceutical ingredient.

The extrudate can comprise at least 20% by weight of the active pharmaceutical ingredient.

The extrudate can comprise at least 25% by weight of the active pharmaceutical ingredient.

The extrudate can comprise at least 30% by weight of the active pharmaceutical ingredient.

The extrudate can comprise from 10% by weight to 50% by weight of the active pharmaceutical ingredient. The extrudate can comprise from 15% by weight to 45% by weight of the active pharmaceutical ingredient. The extrudate can comprise from 20% by weight to 40% by weight of the active pharmaceutical ingredient. The extrudate can comprise from 25% by weight to 40% by weight of the active pharmaceutical ingredient. The extrudate can comprise from 30% by weight to 40% NSAID by weight of the active pharmaceutical ingredient.

Typically, the combination of polymers is miscible with the active pharmaceutical ingredient.

The first polymer of the combination of two polymers can be selected from polymers having a glass transition temperature of at least 30° C. The polymer can have a glass transition temperature of at least 40° C. The polymer can have a glass transition temperature of less than or equal to 60° C. The polymer can have a glass transition temperature of between 40° C. and 50° C.

The first polymer is selected such that it provides stability for the amorphous form of the active pharmaceutical ingredient for a period of at least six months.

Typically, the first polymer of the combination of two polymer can be selected from polymethacrylates including but not limited to amino methacrylate copolymer, amino-methyl methacrylate, methacrylic acid methyl methacrylate, hydroxypropyl methylcellulose, ethyl cellulose, hydroxypropyl methylcellulose acetate succinate, polyethylene glycol, poly(vinylmethyl ether/maleic anhydride), crospovidone, croscarmellose sodium, dimethylaminoethyl methacrylate co-polymer. A preferred polymer is dimethylaminoethyl methacrylate co-polymer.

Alternatively, the first polymer can be selected from a combination of polymers with the proviso that the combination is selected such that it provides stability for the amorphous form of the active pharmaceutical ingredient for a period of at least six months. The first polymer can be a combination of polymers selected from polymethacrylates including but not limited to amino methacrylate copolymer, amino-methyl methacrylate, methacrylic acid methyl methacrylate, hydroxypropyl methylcellulose, ethyl cellulose, hydroxypropyl methylcellulose acetate succinate, polyethylene glycol, poly(vinylmethyl ether/maleic anhydride), crospovidone, croscarmellose sodium, dimethylaminoethyl methacrylate co-polymer.

The first polymer of the combination of two polymers can be present at a level 30-90% by weight of the composition. Preferably the first polymer of the combination of two polymers can be present at a level 40-80% by weight of the composition. More preferably, the first polymer of the combination of two polymers can be present at a level 50-70% by weight of the composition. Most preferably the first polymer of the combination of two polymers can be present at a level of about 60% by weight of the composition.

The second polymer of the combination of two polymers can be selected from polymers having a glass transition temperature of at least 80° C. The polymer can have a glass transition temperature of at least 90° C. The polymer can have a glass transition temperature of less than or equal to 120° C. The polymer can have a glass transition temperature of between 90° C. and 110° C.

Typically, the second polymer of the combination of two polymers can be selected from polyvinylpyrrolidone, polyvinylpyrrolidone-vinyl acetate co-polymer and polyvinyl caprolactam-polyvinyl acetate-polyethylene glycol graft copolymer. Preferably the second polymer is polyvinylpyrrolidone K12 or polyvinylpyrrolidone-vinyl acetate co-polymer (PVPVA64).

Alternatively, the second polymer can be selected from a combination of polymers with the proviso that the combination is selected such that the extrudate has a tensile strength of at least 3 N/mm². The second polymer can be a combination of polymers selected from polyvinylpyrrolidone, polyvinylpyrrolidone-vinyl acetate co-polymer and polyvinyl caprolactam-polyvinyl acetate-polyethylene glycol graft copolymer.

The second polymer is selected such that the extrudate has a tensile strength of 5-50 N/mm².

The second polymer of the combination of two polymers can be present in a level of 1-30% by weight of the composition. Preferably, the second polymer of the combination of two polymers can be present in a level of 10-25% by weight of the composition. More preferably, the second polymer of the combination of two polymers can be present in a level of 15-25% by weight of the composition. Most preferably, the second polymer of the combination of two polymers can be present in a level of about 20% by weight of the composition.

A preferred combination of the first and second polymers is dimethylaminoethyl methacrylate co-polymer and polyvinylpyrrolidone-vinyl acetate co-polymer (PVPVA64). An alternative preferred combination of the first and second polymers is dimethylaminoethyl methacrylate co-polymer and polyvinylpyrrolidone K12.

The ratio of the first polymer and the second polymer of the combination of two polymers can be selected to be from 10:1 to 1:10. A preferred ratio is from 4:1 to 1:4. A more preferred ratio is from 1:1 to 1:3.

The ratio of the active pharmaceutical ingredient, the first polymer and the second polymer can be from 1:1:1 to 1:4:4. Preferably, the ratio of the active pharmaceutical ingredient, the first polymer and the second polymer can be from 1:1.5:1.5 to 1:2.5:2.5. Alternatively, the ratio of the active pharmaceutical ingredient, the first polymer and the second polymer can be from 1:2:1 to 1:4:1.

The active pharmaceutical ingredient is portioned between the each of the first and second polymer of the melt extrudate. Preferably, at least 20% of the active pharmaceutical ingredient is in the first polymer. More preferably at least 25% is in the first polymer. Most preferably at least 30% is in the first polymer. Preferably, less than or equal to 50% of the active pharmaceutical ingredient is in the first polymer. More preferably less than or equal to 40% is in the first polymer. Most preferably less than or equal to 35% is in the first polymer.

Preferably, from 20% by weight to 50% by weight of the active pharmaceutical ingredient is in the first polymer of the melt extrudate. More preferably, from 25% by weight to 40% by weight of the active pharmaceutical ingredient is in the first polymer of the melt extrudate. Most preferably, from 30% by weight to 35% by weight of the active pharmaceutical ingredient is in the first polymer of the melt extrudate.

One or more processing aids such as silicon dioxide, talc, magnesium silicate and glyceryl monostearate can be added prior to melt extrusion.

One or more additional processing aids can be added post-melt extrusion selected from silicon dioxide, microcrystalline cellulose, crospovidone, carrageenan, chitosan, pectinic acid, glycerides, beta-cyclodextrin and cellulose derivatives.

The combination of polymers is soluble at a pH of about 1 to about 5. Preferably, the combination of polymers is soluble at a pH of about 2 to about 4. Alternatively, the combination of polymers is soluble at a pH of about 1 to about 3.

Preferably the difference in Hansen solubility parameter between active pharmaceutical ingredient and the combination of the first polymer and the second polymer is less than 10 MPa^{1/2}. More preferably the difference in Hansen solubility parameter between active pharmaceutical ingredient and the combination of the first polymer and the second polymer is less than 7 MPa^{1/2}.

The extrudate is typically prepared using a hot melt extrusion process.

The melt extrusion process is preferably conducted at a temperature of between 80° C. and 110° C. More preferably, the temperature of the extrusion process is between 90° C. and 100° C. Most preferably, the temperature of the extrusion process is between 93° C. and 98° C.

The screw speed of the extruder can be selected from 1 revolution per minute (rpm) to 30 revolutions per minute (rpm). Preferably the screw speed of the extruder is from 5-20 rpm. More preferably the screw speed of the extruder is about 10 rpm.

The melt extrusion process can be carried out at a preferred temperature from 80° C. to 110° C. and at a preferred extruder screw speed from 1 rpm to 30 rpm. More preferably, the melt extrusion temperature can be between 90° C. and 100° C. and the extruder screw speed can be from 5 rpm to 20 rpm. Most preferably, the melt extrusion temperature can be between 93° C. and 98° C. and the extruder screw speed can be about 10 rpm.

Alternatively, the screw speed of the extruder can be selected from 100 revolutions per minute (rpm) to 300 revolutions per minute (rpm). Preferably the screw speed of the extruder is from 150-250 rpm. Preferably the screw speed of the extruder is from 180-220 rpm. Most preferably the screw speed of the extruder is about 200 rpm.

The melt extrusion process can be carried out at a preferred temperature from 80° C. to 110° C. and at a preferred extruder screw speed from 100 rpm to 300 rpm. More preferably, the melt extrusion temperature can be between 90° C. and 100° C. and the extruder screw speed can be from 150 rpm to 200 rpm. Most preferably, the melt extrusion temperature can be between 93° C. and 98° C. and the extruder screw speed can be about 180-220 rpm. Most preferably the melt extrusion temperature can be about 95° C. and the extruder screw speed can be about 200 rpm.

Alternatively, the melt extrusion process can be carried out at a temperature from 115° C. to 125° C. and the screw speed of the extruder can be selected from 100 revolutions per minute (rpm) to 300 revolutions per minute (rpm). Preferably the melt extrusion process can be carried out at a temperature of about 120° C. and the screw speed of the extruder can be selected to be about 200 rpm.

Preferably the extrusion processing conditions are selected such that the torque is less than or equal to 35 Nm. More preferably the extrusion conditions are selected such that the torque is less than or equal to 25 Nm. Most preferably the extrusion conditions are selected such that the torque is less than or equal to 20 Nm.

Preferably the extrusion conditions are selected such that the torque is more than or equal to 5 Nm. More preferably the extrusion conditions are selected such that the torque is more than or equal to 10 Nm. Most preferably the extrusion conditions are selected such that the torque is more than or equal to 15 Nm.

Preferably the extrusion conditions are selected such that the torque is less than or equal to 35 Nm and more than or equal to 5 Nm. More preferably the extrusion conditions are selected such that the torque is less than or equal to 25 Nm and more than or equal to 10 Nm. Most preferably the extrusion conditions are selected such that the torque is less than or equal to 20 Nm and more than or equal to 15 Nm.

Preferably, the solidified melt extrudate consist essentially of ibuprofen in an amorphous form and a combination of dimethylaminoethyl methacrylate co-polymer and polyvinylpyrrolidone-vinyl acetate co-polymer (PVPVA64) wherein the extrudate can comprise from 30%-40% by weight ibuprofen wherein the extrudate can be prepared using a hot melt extrusion process at a temperature between 93° C. and 98° C. and wherein the extruder screw speed can be about 10 rpm.

According to a third aspect of the present invention there is provided a process for producing solidified melt extrudate granules which contains an active pharmaceutical ingredient in an amorphous form, the process comprising the steps of:
(a) forming a mixture by mixing the active pharmaceutical ingredient and a combination of two or more polymers;
(b) melt-extruding the mixture of the active pharmaceutical ingredient and the combination of two or more polymers;
(c) cooling the extrudate; and
(d) comminuting the melt extrudate.

Preferably, the process incudes a spheronisation step.

The active pharmaceutical ingredient can be selected to be an NSAID or paracetamol.

The NSAID can be selected from ibuprofen, flurbiprofen, ketoprofen, diclofenac, naproxen, aspirin, indomethacin, and meloxicam. A preferred NSAID is ibuprofen.

The extrudate can comprise up to 50% by weight of the active pharmaceutical ingredient.

The extrudate can comprise up to 45% by weight of the active pharmaceutical ingredient.

The extrudate can comprise up to 40% by weight of the active pharmaceutical ingredient.

The extrudate can comprise at least 10% by weight of the active pharmaceutical ingredient.

The extrudate can comprise at least 15% by weight of the active pharmaceutical ingredient.

The extrudate can comprise at least 20% by weight of the active pharmaceutical ingredient.

The extrudate can comprise at least 25% by weight of the active pharmaceutical ingredient.

The extrudate can comprise at least 30% by weight of the active pharmaceutical ingredient.

The extrudate can comprise from 10% by weight to 50% by weight of the active pharmaceutical ingredient. The extrudate can comprise from 15% by weight to 45% by weight of the active pharmaceutical ingredient. The extrudate can comprise from 20% by weight to 40% by weight of the active pharmaceutical ingredient. The extrudate can comprise from 25% by weight to 40% by weight of the active pharmaceutical ingredient. The extrudate can comprise from 30% by weight to 40% NSAID by weight of the active pharmaceutical ingredient.

Typically, the combination of polymers is miscible with the active pharmaceutical ingredient.

The first polymer of the combination of two polymers can be selected from polymers having a glass transition temperature of at least 30° C. The polymer can have a glass transition temperature of at least 40° C. The polymer can have a glass transition temperature of less than or equal to 60° C. The polymer can have a glass transition temperature of between 40° C. and 50° C.

The first polymer is selected such that it provides stability for the amorphous form of the active pharmaceutical ingredient for a period of at least six months.

Typically, the first polymer of the combination of two polymers can be selected from polymethacrylates including but not limited to amino methacrylate copolymer, amino-methyl methacrylate, methacrylic acid methyl methacrylate, hydroxypropyl methylcellulose, ethyl cellulose, hydroxypropyl methylcellulose acetate succinate, polyethylene glycol, poly(vinylmethyl ether/maleic anhydride), crospovidone, croscarmellose sodium, dimethylaminoethyl methacrylate co-polymer. A preferred polymer is dimethylaminoethyl methacrylate co-polymer.

Alternatively, the first polymer can be selected from a combination of polymers with the proviso that the combination is selected such that it provides stability for the amorphous form of the active pharmaceutical ingredient for a period of at least six months. The first polymer can be a combination of polymers selected from polymethacrylates including but not limited to amino methacrylate copolymer, amino-methyl methacrylate, methacrylic acid methyl methacrylate, hydroxypropyl methylcellulose, ethyl cellulose, hydroxypropyl methylcellulose acetate succinate, polyethylene glycol, poly(vinylmethyl ether/maleic anhydride), crospovidone, croscarmellose sodium, dimethylaminoethyl methacrylate co-polymer.

The first polymer of the combination of two polymers can be present at a level 30-90% by weight of the composition. Preferably the first polymer of the combination of two polymers can be present at a level 40-80% by weight of the composition. More preferably, the first polymer of the combination of two polymers can be present at a level 50-70% by weight of the composition. Most preferably the first polymer of the combination of two polymers can be present at a level of about 60% by weight of the composition.

The second polymer of the combination of two polymers can be selected from polymers having a glass transition temperature of at least 80° C. The polymer can have a glass transition temperature of at least 90° C. The polymer can have a glass transition temperature of less than or equal to 120° C. The polymer can have a glass transition temperature of between 90° C. and 110° C.

The second polymer is selected such that the extrudate has a tensile strength of 5-50 N/mm².

Typically, the second polymer of the combination of two polymers can be selected from polyvinylpyrrolidone, polyvinylpyrrolidone-vinyl acetate co-polymer and polyvinyl caprolactam-polyvinyl acetate-polyethylene glycol graft copolymer. Preferably the second polymer is polyvinylpyrrolidone K12 or polyvinylpyrrolidone-vinyl acetate co-polymer (PVPVA64).

Alternatively, the second polymer can be selected from a combination of polymers with the proviso that the combination is selected such that the extrudate has a tensile strength of at least 3 N/mm². The second polymer can be a combination of polymers selected from polyvinylpyrrolidone, polyvinylpyrrolidone-vinyl acetate co-polymer and polyvinyl caprolactam-polyvinyl acetate-polyethylene glycol graft copolymer.

The second polymer of the combination of two polymers can be present in a level of 1-30% by weight of the composition. Preferably, the second polymer of the combination of two polymers can be present in a level of 10-25% by weight of the composition. More preferably, the second polymer of the combination of two polymers can be present in a level of 15-25% by weight of the composition. Most preferably, the second polymer of the combination of two polymers can be present in a level of about 20% by weight of the composition.

A preferred combination of the first and second polymers is dimethylaminoethyl methacrylate co-polymer and polyvinylpyrrolidone-vinyl acetate co-polymer (PVPVA64). An alternative preferred combination of the first and second polymers is dimethylaminoethyl methacrylate co-polymer and polyvinylpyrrolidone K12.

The ratio of the first polymer and the second polymer of the combination of two polymers can be selected to be from 10:1 to 1:10. A preferred ratio is from 4:1 to 1:4. A more preferred ratio is from 1:1 to 1:3.

The ratio of the active pharmaceutical ingredient, the first polymer and the second polymer can be from 1:1:1 to 1:4:4. Preferably, the ratio of the active pharmaceutical ingredient, the first polymer and the second polymer can be from 1:1.5:1.5 to 1:2.5:2.5. Alternatively, the ratio of the active pharmaceutical ingredient, the first polymer and the second polymer can be from 1:2:1 to 1:4:1.

The active pharmaceutical ingredient is portioned between the each of the first and second polymer of the melt extrudate. Preferably, at least 20% of the active pharmaceutical ingredient is in the first polymer. More preferably at least 25% is in the first polymer. Most preferably at least 30% is in the first polymer. Preferably, less than or equal to 50% of the active pharmaceutical ingredient is in the first polymer. More preferably less than or equal to 40% is in the first polymer. Most preferably less than or equal to 35% is in the first polymer.

Preferably, from 20% by weight to 50% by weight of the active pharmaceutical ingredient is in the first polymer of the melt extrudate. More preferably, from 25% by weight to 40% by weight of the active pharmaceutical ingredient is in the first polymer of the melt extrudate. Most preferably, from 30% by weight to 35% by weight of the active pharmaceutical ingredient is in the first polymer of the melt extrudate.

One or more processing aids such as silicon dioxide, talc, magnesium silicate and glyceryl monostearate can be added prior to melt extrusion.

One or more additional processing aids can be added post-melt extrusion selected from silicon dioxide, microcrystalline cellulose, crospovidone, carrageenan, chitosan, pectinic acid, glycerides, beta-cyclodextrin and cellulose derivatives.

The combination of polymers is soluble at a pH of about 1 to about 5. Preferably, the combination of polymers is soluble at a pH of about 2 to about 4. Alternatively, the combination of polymers is soluble at a pH of about 1 to about 3.

Preferably the difference in Hansen solubility parameter between active pharmaceutical ingredient and the combination of the first polymer and the second polymer is less than 10 MPa^{1/2}. More preferably the difference in Hansen solubility parameter between active pharmaceutical ingredient and the combination of the first polymer and the second polymer is less than 7 MPa^{1/2}.

The extrudate granules are typically prepared using a hot melt extrusion process.

The melt extrusion process is preferably conducted at a temperature of between 80° C. and 110° C. More preferably, the temperature of the extrusion process is between 90° C. and 100° C. Most preferably, the temperature of the extrusion process is between 93° C. and 98° C.

The screw speed of the extruder can be selected from 1 revolution per minute (rpm) to 30 revolutions per minute (rpm). Preferably the screw speed of the extruder is from 5-20 rpm. More preferably the screw speed of the extruder is about 10 rpm.

The melt extrusion process can be carried out at a preferred temperature from 80° C. to 110° C. and at a preferred extruder screw speed from 1 rpm to 30 rpm. More preferably, the melt extrusion temperature can be between 90° C. and 100° C. and the extruder screw speed can be from 5 rpm to 20 rpm. Most preferably, the melt extrusion temperature can be between 93° C. and 98° C. and the extruder screw speed can be about 10 rpm.

Alternatively, the screw speed of the extruder can be selected from 100 revolutions per minute (rpm) to 300 revolutions per minute (rpm). Preferably the screw speed of the extruder is from 150-250 rpm. Preferably the screw speed of the extruder is from 180-220 rpm. Most preferably the screw speed of the extruder is about 200 rpm.

The melt extrusion process can be carried out at a preferred temperature from 80° C. to 110° C. and at a preferred extruder screw speed from 100 rpm to 300 rpm. More preferably, the melt extrusion temperature can be between 90° C. and 100° C. and the extruder screw speed can be from 150 rpm to 200 rpm. Most preferably, the melt extrusion temperature can be between 93° C. and 98° C. and the extruder screw speed can be about 180-220 rpm. Most preferably the melt extrusion temperature can be about 95° C. and the extruder screw speed can be about 200 rpm.

Alternatively, the melt extrusion process can be carried out at a temperature from 115° C. to 125° C. and the screw speed of the extruder can be selected from 100 revolutions per minute (rpm) to 300 revolutions per minute (rpm). Preferably the melt extrusion process can be carried out at a temperature of about 120° C. and the screw speed of the extruder can be selected to be about 200 rpm.

Preferably the extrusion processing conditions are selected such that the torque is less than or equal to 35 Nm. More preferably the extrusion conditions are selected such that the torque is less than or equal to 25 Nm. Most preferably the extrusion conditions are selected such that the torque is less than or equal to 20 Nm.

Preferably the extrusion conditions are selected such that the torque is more than or equal to 5 Nm. More preferably the extrusion conditions are selected such that the torque is more than or equal to 10 Nm. Most preferably the extrusion conditions are selected such that the torque is more than or equal to 15 Nm.

Preferably the extrusion conditions are selected such that the torque is less than or equal to 35 Nm and more than or equal to 5 Nm. More preferably the extrusion conditions are selected such that the torque is less than or equal to 25 Nm and more than or equal to 10 Nm. Most preferably the extrusion conditions are selected such that the torque is less than or equal to 20 Nm and more than or equal to 15 Nm.

Preferably, the solidified melt extrudate comprises ibuprofen in an amorphous form and a combination of dimethylaminoethyl methacrylate co-polymer and polyvinylpyrrolidone-vinyl acetate co-polymer (PVPVA64) wherein the extrudate can comprise from 30%-40% by weight ibuprofen wherein the extrudate can be prepared using a hot melt extrusion process at a temperature between 93° C. and 98° C. and wherein the extruder screw speed can be about 10 rpm.

According to a fourth aspect of the present invention there is provided a solidified melt extrudate comprising an NSAID or paracetamol in an amorphous form and a combination of a first polymer having a glass transition temperature of between 40° C. and 50° C. and a second polymer having a glass transition temperature of between 90° C. and 110° C. wherein the extrudate can comprise from 30%-45% by weight NSAID or paracetamol.

The NSAID can be selected from ibuprofen, flurbiprofen, ketoprofen, diclofenac, naproxen, aspirin, indomethacin, and meloxicam. A preferred NSAID is ibuprofen.

Typically, the combination of polymers is miscible with the active pharmaceutical ingredient.

Preferably, the extrudate can be prepared using a hot melt extrusion process at a temperature from about 60° C. to about 120° C. More preferably, the extrudate can be prepared using a hot melt extrusion process at a temperature from about 75° C. to about 95° C. Most preferably, the extrudate can be prepared using a hot melt extrusion process at a temperature from about 80° C. to about 90° C.

The solidified melt extrudate can comprise an NSAID or paracetamol in an amorphous form and a combination of a first polymer having a glass transition temperature of between 40° C. and 50° C. and a second polymer having a glass transition temperature of between 90° C. and 110° C. wherein the extrudate can comprise from 30%-45% by weight NSAID or paracetamol wherein the extrudate can be prepared using a holt melt extrusion process at a temperature from about 75° C. to about 95° C.

The first polymer is selected such that it provides stability for the amorphous form of the active pharmaceutical ingredient for a period of at least six months.

Preferably, the weight ratio of the of the first polymer and the second polymer can be 5:1 to 15:1. More preferably, the weight ratio of the first polymer to the second polymer can be 8:1 to 12:1.

Preferably, the weight ratio of the NSAID, the first polymer and the second polymer can be 5:8:1 to 10:15:1. More preferably, the weight ratio of the NSAID, the first polymer and the second polymer can be 7:10:1 to 9:12:1.

Preferably, the solidified melt extrudate comprises 30%-45% by weight of NSAID in an amorphous form and a combination of 45%-65% by weight of the first polymer and 1%-10% by weight of the second polymer.

Preferably, the solidified melt extrudate comprises 30%-45% by weight of an NSAID in an amorphous form and a combination of 45%-65% by weight of the first polymer and 1%-10% by weight of the second polymer wherein the extrudate can be prepared using a hot melt extrusion process at a temperature from about 75° C. to about 95° C.

Preferably, the solidified melt extrudate comprises 30%-45% by weight of an NSAID in an amorphous form and a combination of 45%-65% by weight of the first polymer and 1%-10% by weight of the second polymer wherein the extrudate can be prepared using a hot melt extrusion process at a temperature from about 75° C. to about 95° C. and wherein the weight ratio of the NSAID, the first polymer and the second polymer is from about 5:8:1 to about 10:15:1. More preferably, the weight ratio of the NSAID, the first polymer and the second polymer is from about 7:10:1 to about 9:12:1.

Preferably, the solidified melt extrudate comprises 30%-45% by weight of an NSAID in an amorphous form and a combination of 45%-65% by weight of the first polymer and 1%-10% by weight of the second polymer wherein the extrudate can be prepared using a hot melt extrusion process at a temperature from about 75° C. to about 95° C. and wherein when the second polymer of the combination of two polymers is selected to be a homopolymer it has an average molecular weight of 1,000 to 5,000 Daltons and when the second polymer of the combination of two polymer is selected to be a co-polymer it has an average molecular weight of from 45,000 to 70,000 Daltons.

Typically, the first polymer of the combination of two polymers can be selected from polymethacrylates including but not limited to amino methacrylate copolymer, amino-methyl methacrylate, methacrylic acid methyl methacrylate, hydroxypropyl methylcellulose, ethyl cellulose, hydroxypropyl methylcellulose acetate succinate, polyethylene glycol, poly(vinylmethyl ether/maleic anhydride), crospovidone, croscarmellose sodium, dimethylaminoethyl methacrylate co-polymer. A preferred polymer is dimethylaminoethyl methacrylate co-polymer.

Alternatively, the first polymer can be selected from a combination of polymers with the proviso that the combination is selected such that it provides stability for the amorphous form of the active pharmaceutical ingredient for a period of at least six months. The first polymer can be a combination of polymers selected from polymethacrylates including but not limited to amino methacrylate copolymer, amino-methyl methacrylate, methacrylic acid methyl methacrylate, hydroxypropyl methylcellulose, ethyl cellulose, hydroxypropyl methylcellulose acetate succinate, polyethylene glycol, poly(vinylmethyl ether/maleic anhydride), crospovidone, croscarmellose sodium, dimethylaminoethyl methacrylate co-polymer.

Preferably, when the second polymer of the combination of two polymers is selected to be a homopolymer it has an average molecular weight of 1,000 to 5,000 Daltons and when the second polymer of the combination of two polymer is selected to be a co-polymer it has an average molecular weight of from 45,000 to 70,000 Daltons.

Typically, the second polymer of the combination of two polymers can be selected from polyvinylpyrrolidone, polyvinylpyrrolidone-vinyl acetate co-polymer and polyvinyl caprolactam-polyvinyl acetate-polyethylene glycol graft copolymer. Preferably the second polymer is polyvinylpyrrolidone K12 or polyvinylpyrrolidone-vinyl acetate co-polymer (PVPVA64).

The second polymer is selected such that the extrudate has a tensile strength of 5-50 N/mm².

The active pharmaceutical ingredient is portioned between the each of the first and second polymer of the melt extrudate. Preferably, at least 20% of the of the active pharmaceutical ingredient is in the first polymer. More preferably at least 25% is in the first polymer. Most preferably at least 30% is in the first polymer. Preferably, less than or equal to 50% of the active pharmaceutical ingredient is in the first polymer. More preferably less than or equal to 40% is in the first polymer. Most preferably less than or equal to 35% is in the first polymer.

Preferably, from 20% by weight to 50% by weight of the active pharmaceutical ingredient is in the first polymer of the melt extrudate. More preferably, from 25% by weight to 40% by weight of the active pharmaceutical ingredient is in the first polymer of the melt extrudate. Most preferably, from 30% by weight to 35% by weight of the active pharmaceutical ingredient is in the first polymer of the melt extrudate.

One or more processing aids such as silicon dioxide, talc, magnesium silicate and glyceryl monostearate can be added prior to melt extrusion.

One or more additional processing aids can be added post-melt extrusion selected from silicon dioxide, microcrystalline cellulose, crospovidone, carrageenan, chitosan, pectinic acid, glycerides, beta-cyclodextrin and cellulose derivatives.

The combination of polymers is soluble at a pH of about 1 to about 5. Preferably, the combination of polymers is soluble at a pH of about 1 to about 3.

Preferably the difference in Hansen solubility parameter between active pharmaceutical ingredient and the combination of the first polymer and the second polymer is less than 10 MPa^{1/2}. More preferably the difference in Hansen solubility parameter between active pharmaceutical ingredient and the combination of the first polymer and the second polymer is less than 7 MPa^{1/2}.

Preferably, the solidified melt extrudate comprises an NSAID in an amorphous form and a combination of dimethylaminoethyl methacrylate co-polymer and polyvinylpyrrolidone-vinyl acetate co-polymer (PVPVA64) wherein the extrudate can comprise from 30%-45% by weight NSAID wherein the extrudate can be prepared using a hot melt extrusion process at a temperature from about 75° C. to about 95° C.

Preferably, the weight ratio of the dimethylaminoethyl methacrylate co-polymer and polyvinylpyrrolidone-vinyl acetate co-polymer (PVPVA64) can be 5:1 to 15:1. More preferably, the weight ratio of the first polymer is 8:1 to 12:1.

Preferably, the weight ratio of the NSAID, the dimethylaminoethyl methacrylate co-polymer and the polyvinylpyrrolidone-vinyl acetate co-polymer (PVPVA64) can be 5:8:1 to 10:15:1. More preferably, the weight ratio of the NSAID, the dimethylaminoethyl methacrylate co-polymer and the polyvinylpyrrolidone-vinyl acetate co-polymer (PVPVA64) can be 7:10:1 to 9:12:1.

Preferably, the solidified melt extrudate comprises 30%-45% by weight of NSAID in an amorphous form and a combination of 45%-65% by weight of dimethylaminoethyl methacrylate co-polymer and 1%-10% by weight of polyvinylpyrrolidone-vinyl acetate co-polymer (PVPVA64).

Preferably, the solidified melt extrudate comprises 30%-45% by weight of NSAID in an amorphous form and a combination of 45%-65% by weight of dimethylaminoethyl methacrylate co-polymer and 1%-10% by weight of polyvinylpyrrolidone-vinyl acetate co-polymer (PVPVA64) wherein the extrudate can be prepared using a hot melt extrusion process at a temperature from about 75° C. to about 95° C.

Preferably, the solidified melt extrudate comprises 30%-45% by weight of an NSAID in an amorphous form and a combination of 45%-65% by weight of dimethylaminoethyl methacrylate co-polymer and 1%-10% by weight of polyvinylpyrrolidone-vinyl acetate co-polymer (PVPVA64) wherein the extrudate can be prepared using a hot melt extrusion process at a temperature from about 75° C. to about 95° C. and wherein the weight ratio of the NSAID, the dimethylaminoethyl methacrylate co-polymer and the polyvinylpyrrolidone-vinyl acetate co-polymer (PVPVA64) is from about 5:8:1 to about 10:15:1. More preferably, the weight ratio of the NSAID, the dimethylaminoethyl methacrylate co-polymer and the polyvinylpyrrolidone-vinyl acetate co-polymer (PVPVA64) is from about 7:10:1 to about 9:12:1.

Preferably, the solidified melt extrudate comprises ibuprofen in an amorphous form and a combination of a first polymer having a glass transition temperature of between 40° C. and 50° C. and a second polymer having a glass transition temperature of between 90° C. and 110° C. wherein the extrudate can comprise from 30%-45% by weight of ibuprofen. The extrudate can be prepared using a hot melt extrusion process at a temperature from about 75° C. to about 95° C.

Typically, the combination of polymers is miscible with the active pharmaceutical ingredient.

The first polymer is selected such that it provides stability for the amorphous form of the active pharmaceutical ingredient for a period of at least six months.

Preferably, when the second polymer of the combination of two polymers is selected to be a homopolymer it has an average molecular weight of 1,000 to 5,000 Daltons and when the second polymer of the combination of two polymers is selected to be a co-polymer it has an average molecular weight of from 45,000 to 70,000 Daltons.

Preferably, the weight ratio of the of the first polymer and the second polymer can be 5:1 to 15:1. More preferably, the weight ratio of the first polymer to the second polymer can be 8:1 to 12:1.

Preferably, the weight ratio of the ibuprofen, the first polymer and the second polymer can be 5:8:1 to 10:15:1. More preferably, the weight ratio of the ibuprofen, the first polymer and the second polymer can be 7:10:1 to 9:12:1.

Preferably, the solidified melt extrudate comprises 30%-45% by weight of ibuprofen in an amorphous form and a combination of 45%-65% by weight of the first polymer and 1%-10% by weight of the second polymer.

Preferably, the solidified melt extrudate comprises 30%-45% by weight of ibuprofen in an amorphous form and a combination of 45%-65% by weight of the first polymer and 1%-10% by weight of the second polymer wherein the extrudate can be prepared using a hot melt extrusion process at a temperature from about 75° C. to about 95° C.

Preferably, the solidified melt extrudate comprises 30%-45% by weight of ibuprofen in an amorphous form and a combination of 45%-65% by weight of the first polymer and 1%-10% by weight of the second polymer wherein the extrudate can be prepared using a hot melt extrusion process at a temperature from about 75° C. to about 95° C. and wherein the weight ratio of the ibuprofen, the first polymer and the second polymer is from about 5:8:1 to about 10:15:1. More preferably, the weight ratio of the ibuprofen, the first polymer and the second polymer is from about 7:10:1 to about 9:12:1.

Preferably, the solidified melt extrudate comprises 30%-45% by weight of ibuprofen in an amorphous form and a combination of 45%-65% by weight of the first polymer and 1%-10% by weight of the second polymer wherein the extrudate can be prepared using a hot melt extrusion process at a temperature from about 75° C. to about 95° C. and wherein when the second polymer of the combination of two polymers is selected to be a homopolymer it has an average molecular weight of 1,000 to 5,000 Daltons and when the second polymer of the combination of two polymer is selected to be a co-polymer it has an average molecular weight of from 45,000 to 70,000 Daltons.

Typically, the first polymer of the combination of two polymers can be selected from polymethacrylates including but not limited to amino methacrylate copolymer, amino-methyl methacrylate, methacrylic acid methyl methacrylate, hydroxypropyl methylcellulose, ethyl cellulose, hydroxypropyl methylcellulose acetate succinate, polyethylene glycol, poly(vinylmethyl ether/maleic anhydride), crospovidone, croscarmellose sodium, dimethylaminoethyl methacrylate co-polymer. A preferred polymer is dimethylaminoethyl methacrylate co-polymer.

Alternatively, the first polymer can be selected from a combination of polymers with the proviso that the combination is selected such that it provides stability for the amorphous form of the active pharmaceutical ingredient for a period of at least six months. The first polymer can be a combination of polymers selected from polymethacrylates including but not limited to amino methacrylate copolymer, amino-methyl methacrylate, methacrylic acid methyl methacrylate, hydroxypropyl methylcellulose, ethyl cellulose, hydroxypropyl methylcellulose acetate succinate, polyethylene glycol, poly(vinylmethyl ether/maleic anhydride), crospovidone, croscarmellose sodium, dimethylaminoethyl methacrylate co-polymer.

Typically, the second polymer of the combination of two polymers can be selected from polyvinylpyrrolidone, polyvinylpyrrolidone-vinyl acetate co-polymer and polyvinyl caprolactam-polyvinyl acetate-polyethylene glycol graft copolymer. Preferably the second polymer is polyvinylpyrrolidone K12 or polyvinylpyrrolidone-vinyl acetate co-polymer (PVPVA64).

The second polymer is selected such that the extrudate has a tensile strength of 5-50 N/mm².

The active pharmaceutical ingredient is portioned between the each of the first and second polymer of the melt extrudate. Preferably, at least 20% of the of the active pharmaceutical ingredient is in the first polymer. More preferably at least 25% is in the first polymer. Most preferably at least 30% is in the first polymer. Preferably, less than or equal to 50% of the active pharmaceutical ingredient is in the first polymer. More preferably less than or equal to 40% is in the first polymer. Most preferably less than or equal to 35% is in the first polymer.

Preferably, from 20% by weight to 50% by weight of the active pharmaceutical ingredient is in the first polymer of the melt extrudate. More preferably, from 25% by weight to 40% by weight of the active pharmaceutical ingredient is in the first polymer of the melt extrudate. Most preferably, from 30% by weight to 35% by weight of the active pharmaceutical ingredient is in the first polymer of the melt extrudate.

One or more processing aids such as silicon dioxide, talc, magnesium silicate and glyceryl monostearate can be added prior to melt extrusion.

One or more additional processing aids can be added post-melt extrusion selected from silicon dioxide, microcrystalline cellulose, crospovidone, carrageenan, chitosan, pectinic acid, glycerides, beta-cyclodextrin and cellulose derivatives.

The combination of polymers is soluble at a pH of about 1 to about 5. Preferably, the combination of polymers is soluble at a pH of about 1 to about 3.

Preferably the difference in Hansen solubility parameter between active pharmaceutical ingredient and the combination of the first polymer and the second polymer is less than 10 MPa^{1/2}. More preferably the difference in Hansen solubility parameter between active pharmaceutical ingredient and the combination of the first polymer and the second polymer is less than 7 MPa^{1/2}.

Preferably, the solidified melt extrudate comprises ibuprofen in an amorphous form and a combination of dimethylaminoethyl methacrylate co-polymer and polyvinylpyrrolidone-vinyl acetate co-polymer (PVPVA64) wherein the extrudate can comprise from 30%-45% by weight of ibuprofen wherein the extrudate can be prepared using a hot melt extrusion process at a temperature from about 75° C. to about 95° C.

Preferably, the weight ratio of the dimethylaminoethyl methacrylate co-polymer and polyvinylpyrrolidone-vinyl acetate co-polymer (PVPVA64) can be 5:1 to 15:1. More preferably, the weight ratio of the first polymer is 8:1 to 12:1.

Preferably, the weight ratio of the NSAID, the dimethylaminoethyl methacrylate co-polymer and the polyvinylpyrrolidone-vinyl acetate co-polymer (PVPVA64) can be 5:8:1 to 10:15:1. More preferably, the weight ratio of the NSAID, the dimethylaminoethyl methacrylate co-polymer and the polyvinylpyrrolidone-vinyl acetate co-polymer (PVPVA64) can be 7:10:1 to 9:12:1.

Preferably, the solidified melt extrudate comprises 30%-45% by weight of ibuprofen in an amorphous form and a combination of 45%-65% by weight of dimethylaminoethyl methacrylate co-polymer and 1%-10% by weight the polyvinylpyrrolidone-vinyl acetate co-polymer (PVPVA64).

Preferably, the solidified melt extrudate comprises 30%-45% by weight of ibuprofen in an amorphous form and a combination of 45%-65% by weight of dimethylaminoethyl methacrylate co-polymer and 1%-10% by weight the polyvinylpyrrolidone-vinyl acetate co-polymer (PVPVA64) wherein the extrudate can be prepared using a hot melt extrusion process at a temperature from about 75° C. to about 95° C.

Preferably, the solidified melt extrudate comprises 30%-45% by weight of ibuprofen in an amorphous form and a combination of 45%-65% by weight of dimethylaminoethyl methacrylate co-polymer and 1%-10% by weight of polyvinylpyrrolidone-vinyl acetate co-polymer (PVPVA64) wherein the extrudate can be prepared using a hot melt extrusion process at a temperature from about 75° C. to about 95° C. and wherein the weight ratio of the ibuprofen, the dimethylaminoethyl methacrylate co-polymer and the polyvinylpyrrolidone-vinyl acetate co-polymer (PVPVA64) is from about 5:8:1 to about 10:15:1. More preferably, the weight ratio of the ibuprofen, the dimethylaminoethyl methacrylate co-polymer and the polyvinylpyrrolidone-vinyl acetate co-polymer (PVPVA64) is from about 7:10:1 to about 9:12:1.

According to a fifth aspect of the present invention there is provided a solidified melt extrudate consisting essentially of an NSAID or paracetamol in an amorphous form and a combination of a first polymer having a glass transition temperature of between 40° C. and 50° C. and a second polymer having a glass transition temperature of between 90° C. and 110° C. wherein the extrudate can comprise from 30%-45% by weight NSAID.

The NSAID can be selected from ibuprofen, flurbiprofen, ketoprofen, diclofenac, naproxen, aspirin, indomethacin, and meloxicam. A preferred NSAID is ibuprofen.

Preferably, the extrudate can be prepared using a hot melt extrusion process at a temperature from about 60° C. to about 120° C. More preferably, the extrudate can be prepared using a hot melt extrusion process at a temperature from about 75° C. to about 95° C. Most preferably, the extrudate can be prepared using a hot melt extrusion process at a temperature from about 80° C. to about 90° C.

Typically, the combination of polymers is miscible with the active pharmaceutical ingredient.

The first polymer is selected such that it provides stability for the amorphous form of the active pharmaceutical ingredient for a period of at least six months.

Preferably, when the second polymer of the combination of two polymers is selected to be a homopolymer it has an average molecular weight of 1,000 to 5,000 Daltons and when the second polymer of the combination of two polymer is selected to be a co-polymer it has an average molecular weight of from 45,000 to 70,000 Daltons.

Preferably, the weight ratio of the of the first polymer and the second polymer can be 5:1 to 15:1. More preferably, the weight ratio of the first polymer to the second polymer can be 8:1 to 12:1.

Preferably, the weight ratio of the NSAID, the first polymer and the second polymer can be 5:8:1 to 10:15:1. More preferably, the weight ratio of the NSAID, the first polymer and the second polymer can be 7:10:1 to 9:12:1.

Preferably, the solidified melt extrudate comprises 30%-45% by weight of NSAID in an amorphous form and a combination of 45%-65% by weight of the first polymer and 1%-10% by weight of the second polymer.

Preferably, the solidified melt extrudate comprises 30%-45% by weight of an NSAID in an amorphous form and a combination of 45%-65% by weight of the first polymer and 1%-10% by weight of the second polymer wherein the extrudate can be prepared using a hot melt extrusion process at a temperature from about 75° C. to about 95° C.

Preferably, the solidified melt extrudate comprises 30%-45% by weight of an NSAID in an amorphous form and a combination of 45%-65% by weight of the first polymer and 1%-10% by weight of the second polymer wherein the extrudate can be prepared using a hot melt extrusion process at a temperature from about 75° C. to about 95° C. and wherein the weight ratio of the NSAID, the first polymer and the second polymer is from about 5:8:1 to about 10:15:1. More preferably, the weight ratio of the NSAID, the first polymer and the second polymer is from about 7:10:1 to about 9:12:1.

Preferably, the solidified melt extrudate comprises 30%-45% by weight of an NSAID in an amorphous form and a combination of 45%-65% by weight of the first polymer and 1%-10% by weight of the second polymer wherein the extrudate can be prepared using a hot melt extrusion process at a temperature from about 75° C. to about 95° C. and wherein when the second polymer of the combination of two polymers is selected to be a homopolymer it has an average molecular weight of 1,000 to 5,000 Daltons and when the second polymer of the combination of two polymer is selected to be a co-polymer it has an average molecular weight of from 45,000 to 70,000 Daltons.

Typically, the first polymer of the combination of two polymers can be selected from polymethacrylates including but not limited to amino methacrylate copolymer, amino-methyl methacrylate, methacrylic acid methyl methacrylate, hydroxypropyl methylcellulose, ethyl cellulose, hydroxypropyl methylcellulose acetate succinate, polyethylene glycol, poly(vinylmethyl ether/maleic anhydride), crospovidone, croscarmellose sodium, dimethylaminoethyl methacrylate co-polymer. A preferred polymer is dimethylaminoethyl methacrylate co-polymer.

Alternatively, the first polymer can be selected from a combination of polymers with the proviso that the combination is selected such that it provides stability for the amorphous form of the active pharmaceutical ingredient for a period of at least six months. The first polymer can be a combination of polymers selected from polymethacrylates including but not limited to amino methacrylate copolymer, amino-methyl methacrylate, methacrylic acid methyl methacrylate, hydroxypropyl methylcellulose, ethyl cellulose, hydroxypropyl methylcellulose acetate succinate, polyethylene glycol, poly(vinylmethyl ether/maleic anhydride), crospovidone, croscarmellose sodium, dimethylaminoethyl methacrylate co-polymer.

Typically, the second polymer of the combination of two polymers can be selected from polyvinylpyrrolidone, polyvinylpyrrolidone-vinyl acetate co-polymer and polyvinyl caprolactam-polyvinyl acetate-polyethylene glycol graft copolymer. Preferably the second polymer is polyvinylpyrrolidone K12 or polyvinylpyrrolidone-vinyl acetate co-polymer (PVPVA64).

The second polymer is selected such that the extrudate has a tensile strength of 5-50 N/mm².

The active pharmaceutical ingredient is portioned between the each of the first and second polymer of the melt extrudate. Preferably, at least 20% of the of the active pharmaceutical ingredient is in the first polymer. More preferably at least 25% is in the first polymer. Most preferably at least 30% is in the first polymer. Preferably, less than or equal to 50% of the active pharmaceutical ingredient is in the first polymer. More preferably less than or equal to 40% is in the first polymer. Most preferably less than or equal to 35% is in the first polymer.

Preferably, from 20% by weight to 50% by weight of the active pharmaceutical ingredient is in the first polymer of the melt extrudate. More preferably, from 25% by weight to 40% by weight of the active pharmaceutical ingredient is in the first polymer of the melt extrudate. Most preferably, from 30% by weight to 35% by weight of the active pharmaceutical ingredient is in the first polymer of the melt extrudate.

One or more processing aids such as silicon dioxide, talc, magnesium silicate and glyceryl monostearate can be added prior to melt extrusion.

One or more additional processing aids can be added post-melt extrusion selected from silicon dioxide, microcrystalline cellulose, crospovidone, carrageenan, chitosan, pectinic acid, glycerides, beta-cyclodextrin and cellulose derivatives.

The combination of polymers is soluble at a pH of about 1 to about 5. Preferably, the combination of polymers is soluble at a pH of about 1 to about 3.

Preferably the difference in Hansen solubility parameter between active pharmaceutical ingredient and the combination of the first polymer and the second polymer is less than 10 MPa^{1/2}. More preferably the difference in Hansen solubility parameter between active pharmaceutical ingredient and the combination of the first polymer and the second polymer is less than 7 MPa^{1/2}.

Preferably, the solidified melt extrudate comprises an NSAID in an amorphous form and a combination of dimethylaminoethyl methacrylate co-polymer and polyvinylpyrrolidone-vinyl acetate co-polymer (PVPVA64) wherein the extrudate can comprise from 30%-45% by weight NSAID wherein the extrudate can be prepared using a hot melt extrusion process at a temperature from about 75° C. to about 95° C.

Preferably, the weight ratio of the dimethylaminoethyl methacrylate co-polymer and polyvinylpyrrolidone-vinyl acetate co-polymer (PVPVA64) can be 5:1 to 15:1. More preferably, the weight ratio of the first polymer is 8:1 to 12:1.

Preferably, the weight ratio of the NSAID, the dimethylaminoethyl methacrylate co-polymer and the polyvinylpyrrolidone-vinyl acetate co-polymer (PVPVA64) can be 5:8:1 to 10:15:1. More preferably, the weight ratio of the NSAID, the dimethylaminoethyl methacrylate co-polymer and the polyvinylpyrrolidone-vinyl acetate co-polymer (PVPVA64) can be 7:10:1 to 9:12:1.

Preferably, the solidified melt extrudate comprises 30%-45% by weight of NSAID in an amorphous form and a combination of 45%-65% by weight of dimethylaminoethyl methacrylate co-polymer and 1%-10% by weight the polyvinylpyrrolidone-vinyl acetate co-polymer (PVPVA64).

Preferably, the solidified melt extrudate comprises 30%-45% by weight of NSAID in an amorphous form and a combination of 45%-65% by weight of dimethylaminoethyl methacrylate co-polymer and 1%-10% by weight of polyvinylpyrrolidone-vinyl acetate co-polymer (PVPVA64) wherein the extrudate can be prepared using a hot melt extrusion process at a temperature from about 75° C. to about 95° C.

Preferably, the solidified melt extrudate comprises 30%-45% by weight of an NSAID in an amorphous form and a combination of 45%-65% by weight of dimethylaminoethyl methacrylate co-polymer and 1%-10% by weight of polyvinylpyrrolidone-vinyl acetate co-polymer (PVPVA64) wherein the extrudate can be prepared using a hot melt extrusion process at a temperature from about 75° C. to about 95° C. and wherein the weight ratio of the NSAID, the dimethylaminoethyl methacrylate co-polymer and the polyvinylpyrrolidone-vinyl acetate co-polymer (PVPVA64) is from about 5:8:1 to about 10:15:1. More preferably, the weight ratio of the NSAID, the dimethylaminoethyl methacrylate co-polymer and the polyvinylpyrrolidone-vinyl acetate co-polymer (PVPVA64) is from about 7:10:1 to about 9:12:1.

Preferably, the solidified melt extrudate comprises ibuprofen in an amorphous form and a combination of a first polymer having a glass transition temperature of between 40° C. and 50° C. and a second polymer having a glass transition temperature of between 90° C. and 110° C. wherein the extrudate can comprise from 30%-45% by weight of ibuprofen wherein the extrudate can be prepared using a hot melt extrusion process at a temperature from about 75° C. to about 95° C.

Typically, the combination of polymers is miscible with the active pharmaceutical ingredient.

The first polymer is selected such that it provides stability for the amorphous form of the active pharmaceutical ingredient for a period of at least six months.

Preferably, when the second polymer of the combination of two polymers is selected to be a homopolymer it has an average molecular weight of 1,000 to 5,000 Daltons and when the second polymer of the combination of two polymers is selected to be a co-polymer it has an average molecular weight of from 45,000 to 70,000 Daltons.

Preferably, the weight ratio of the of the first polymer and the second polymer can be 5:1 to 15:1. More preferably, the weight ratio of the first polymer to the second polymer can be 8:1 to 12:1.

Preferably, the weight ratio of the ibuprofen, the first polymer and the second polymer can be 5:8:1 to 10:15:1. More preferably, the weight ratio of the ibuprofen, the first polymer and the second polymer can be 7:10:1 to 9:12:1.

Preferably, the solidified melt extrudate comprises 30%-45% by weight of ibuprofen in an amorphous form and a combination of 45%-65% by weight of the first polymer and 1%-10% by weight of the second polymer.

Preferably, the solidified melt extrudate comprises 30%-45% by weight of ibuprofen in an amorphous form and a combination of 45%-65% by weight of the first polymer and 1%-10% by weight of the second polymer wherein the extrudate can be prepared using a hot melt extrusion process at a temperature from about 75° C. to about 95° C.

Preferably, the solidified melt extrudate comprises 30%-45% by weight of ibuprofen in an amorphous form and a combination of 45%-65% by weight of the first polymer and 1%-10% by weight of the second polymer wherein the extrudate can be prepared using a hot melt extrusion process at a temperature from about 75° C. to about 95° C. and wherein the weight ratio of the ibuprofen, the first polymer and the second polymer is from about 5:8:1 to about 10:15:1. More preferably, the weight ratio of the ibuprofen, the first polymer and the second polymer is from about 7:10:1 to about 9:12:1.

Preferably, the solidified melt extrudate comprises 30%-45% by weight of ibuprofen in an amorphous form and a combination of 45%-65% by weight of the first polymer and 1%-10% by weight of the second polymer wherein the extrudate can be prepared using a hot melt extrusion process at a temperature from about 75° C. to about 95° C. and wherein when the second polymer of the combination of two polymers is selected to be a homopolymer it has an average molecular weight of 1,000 to 5,000 Daltons and when the second polymer of the combination of two polymer is selected to be a co-polymer it has an average molecular weight of from 45,000 to 70,000 Daltons.

Typically, the first polymer of the combination of two polymers can be selected from polymethacrylates including but not limited to amino methacrylate copolymer, amino-methyl methacrylate, methacrylic acid methyl methacrylate, hydroxypropyl methylcellulose, ethyl cellulose, hydroxypropyl methylcellulose acetate succinate, polyethylene glycol, poly(vinylmethyl ether/maleic anhydride), crospovidone, croscarmellose sodium, dimethylaminoethyl methacrylate co-polymer. A preferred polymer is dimethylaminoethyl methacrylate co-polymer.

Alternatively, the first polymer can be selected from a combination of polymers with the proviso that the combination is selected such that it provides stability for the amorphous form of the active pharmaceutical ingredient for a period of at least six months. The first polymer can be a combination of polymers selected from polymethacrylates including but not limited to amino methacrylate copolymer, amino-methyl methacrylate, methacrylic acid methyl methacrylate, hydroxypropyl methylcellulose, ethyl cellulose, hydroxypropyl methylcellulose acetate succinate, polyethylene glycol, poly(vinylmethyl ether/maleic anhydride), crospovidone, croscarmellose sodium, dimethylaminoethyl methacrylate co-polymer.

Typically, the second polymer of the combination of two polymers can be selected from polyvinylpyrrolidone, polyvinylpyrrolidone-vinyl acetate co-polymer and polyvinyl caprolactam-polyvinyl acetate-polyethylene glycol graft copolymer. Preferably the second polymer is polyvinylpyrrolidone K12 or polyvinylpyrrolidone-vinyl acetate co-polymer (PVPVA64).

The second polymer is selected such that the extrudate has a tensile strength of 5-50 N/mm².

The active pharmaceutical ingredient is portioned between the each of the first and second polymer of the melt extrudate. Preferably, at least 20% of the of the active pharmaceutical ingredient is in the first polymer. More preferably at least 25% is in the first polymer. Most preferably at least 30% is in the first polymer. Preferably, less than or equal to 50% of the active pharmaceutical ingredient is in the first polymer. More preferably less than or equal to 40% is in the first polymer. Most preferably less than or equal to 35% is in the first polymer.

Preferably, from 20% by weight to 50% by weight of the active pharmaceutical ingredient is in the first polymer of the melt extrudate. More preferably, from 25% by weight to 40% by weight of the active pharmaceutical ingredient is in the first polymer of the melt extrudate. Most preferably, from 30% by weight to 35% by weight of the active pharmaceutical ingredient is in the first polymer of the melt extrudate.

One or more processing aids such as silicon dioxide, talc, magnesium silicate and glyceryl monostearate can be added prior to melt extrusion.

One or more additional processing aids can be added post-melt extrusion selected from silicon dioxide, microcrystalline cellulose, crospovidone, carrageenan, chitosan, pectinic acid, glycerides, beta-cyclodextrin and cellulose derivatives.

The combination of polymers is soluble at a pH of about 1 to about 5. Preferably, the combination of polymers is soluble at a pH of about 1 to about 3.

Preferably the difference in Hansen solubility parameter between active pharmaceutical ingredient and the combination of the first polymer and the second polymer is less than 10 MPa^{1/2}. More preferably the difference in Hansen solubility parameter between active pharmaceutical ingredient and the combination of the first polymer and the second polymer is less than 7 MPa^{1/2}.

Preferably, the solidified melt extrudate comprises ibuprofen in an amorphous form and a combination of dimethylaminoethyl methacrylate co-polymer and polyvinylpyrrolidone-vinyl acetate co-polymer (PVPVA64) wherein the extrudate can comprise from 30%-45% by weight of ibuprofen. The extrudate can be prepared using a hot melt extrusion process at a temperature from about 75° C. to about 95° C.

Preferably, the weight ratio of the dimethylaminoethyl methacrylate co-polymer and polyvinylpyrrolidone-vinyl acetate co-polymer (PVPVA64) can be 5:1 to 15:1. More preferably, the weight ratio of the first polymer is 8:1 to 12:1.

Preferably, the weight ratio of the NSAID, the dimethylaminoethyl methacrylate co-polymer and the polyvinylpyrrolidone-vinyl acetate co-polymer (PVPVA64) can be 5:8:1 to 10:15:1. More preferably, the weight ratio of the NSAID, the dimethylaminoethyl methacrylate co-polymer and the polyvinylpyrrolidone-vinyl acetate co-polymer (PVPVA64) can be 7:10:1 to 9:12:1.

Preferably, the solidified melt extrudate comprises 30%-45% by weight of ibuprofen in an amorphous form and a combination of 45%-65% by weight of dimethylaminoethyl methacrylate co-polymer and 1%-10% by weight of polyvinylpyrrolidone-vinyl acetate co-polymer (PVPVA64).

Preferably, the solidified melt extrudate comprises 30%-45% by weight of ibuprofen in an amorphous form and a combination of 45%-65% by weight of dimethylaminoethyl methacrylate co-polymer and 1%-10% by weight of polyvinylpyrrolidone-vinyl acetate co-polymer (PVPVA64) wherein the extrudate can be prepared using a hot melt extrusion process at a temperature from about 75° C. to about 95° C.

Preferably, the solidified melt extrudate comprises 30%-45% by weight of ibuprofen in an amorphous form and a combination of 45%-65% by weight of dimethylaminoethyl methacrylate co-polymer and 1%-10% by weight of polyvinylpyrrolidone-vinyl acetate co-polymer (PVPVA64) wherein the extrudate can be prepared using a hot melt extrusion process at a temperature from about 75° C. to about 95° C. and wherein the weight ratio of the ibuprofen, the dimethylaminoethyl methacrylate co-polymer and the polyvinylpyrrolidone-vinyl acetate co-polymer (PVPVA64) is from about 5:8:1 to about 10:15:1. More preferably, the weight ratio of the ibuprofen, the dimethylaminoethyl methacrylate co-polymer and the polyvinylpyrrolidone-vinyl acetate co-polymer (PVPVA64) is from about 7:10:1 to about 9:12:1.

For the avoidance of doubt the term “amorphous” has the meaning that the ibuprofen has no defined crystalline structure characteristic of conventional ibuprofen.

For the avoidance of doubt the active pharmaceutical ingredient can be in either a crystalline form or an amorphous form prior to the melt extrusion process.

In the context of the present invention the terms ‘granule’ and ‘granules’ as used herein refer to discrete particle or particles and includes pellets, powders or spheres.

The amount of ibuprofen in each polymer was measured using infrared spectroscopy of the extrudate prior to any further processing steps.

It will be appreciated that all of the features of the first aspect of the present invention represent preferred features of all other aspects of the present invention. Similarly, all of the features of a particular aspect of the present invention represent preferred features of all other aspects of the present invention.

Embodiments of the invention will now be described, by way of example only, in which:

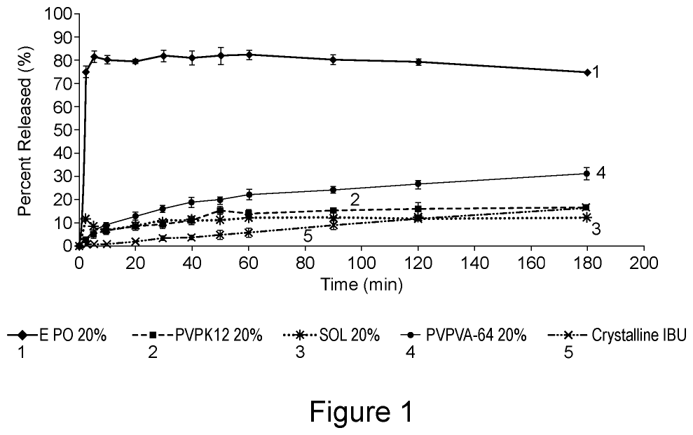

FIG. 1 is a graph comparing the dissolution profile of amorphous ibuprofen from compositions based on a single polymer carrier;

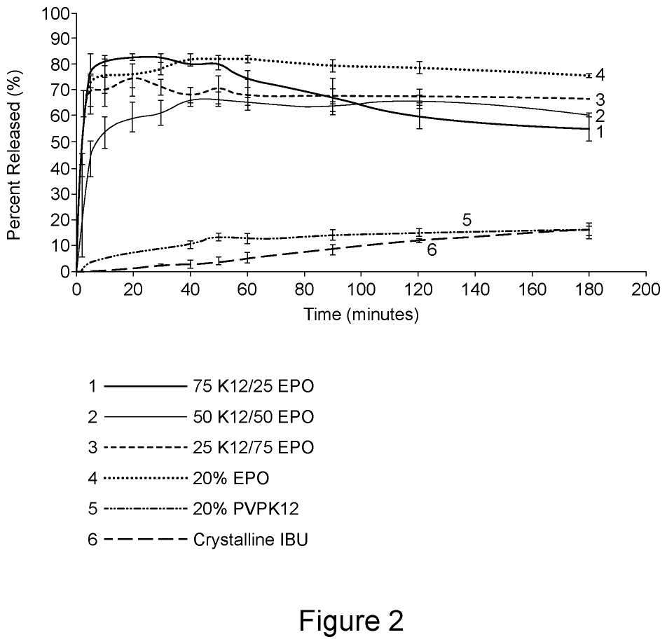

FIGS. 2 & 3 are graphs comparing the dissolution profile for some example embodiments of the present invention extruded at 95° C. and a screw speed of 10 rpm with compositions that fall outwith the scope of the invention for comparison; and

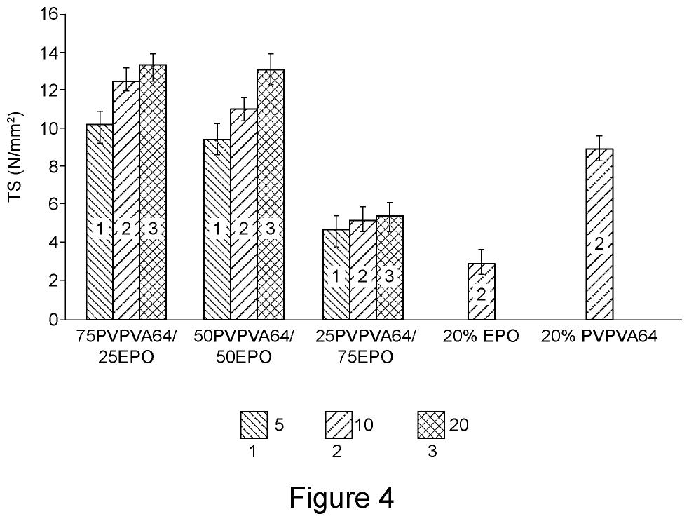

FIG. 4 is a graph comparing the tensile strength of some example embodiments of the present invention with a composition which uses only Eudragit (EPO).

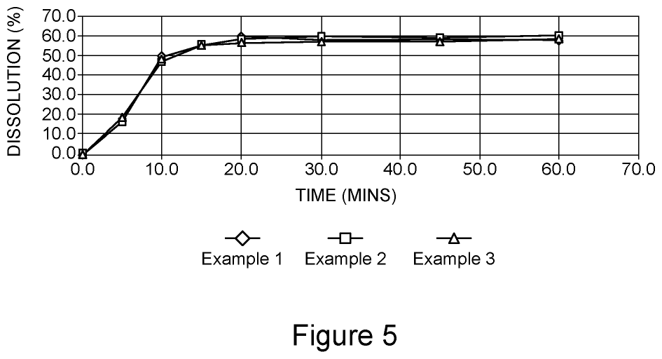

FIG. 5 is a graph comparing the dissolution profile for some additional example embodiments of the present invention;

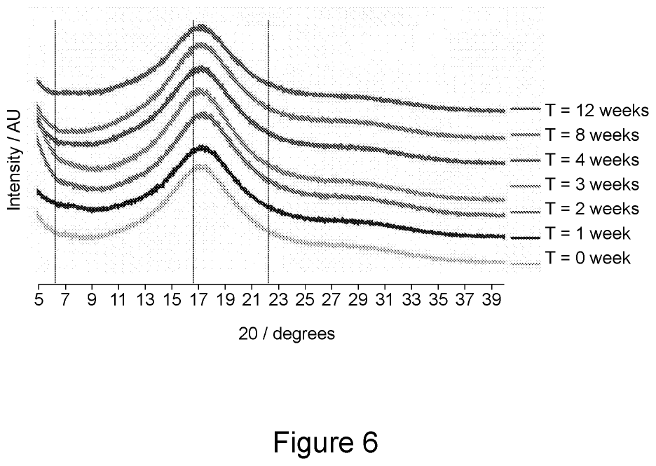

FIG. 6 shows PXRD patterns of compositions of the present invention stored at room temperature over a period of 12 weeks; and

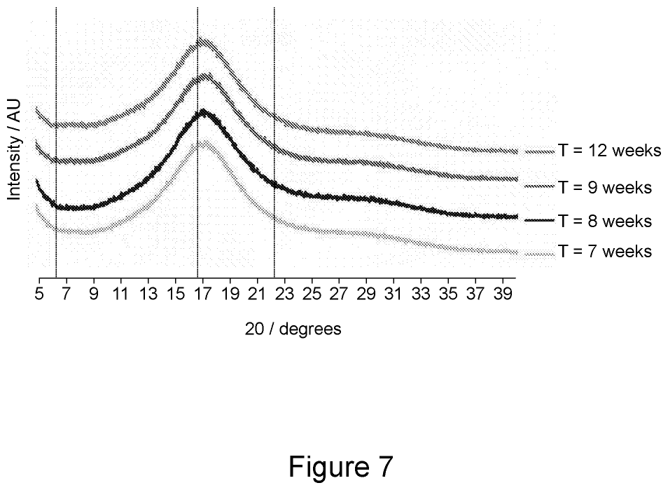

FIG. 7 shows PXRD patterns of compositions of the present invention stored at 40° C. over a period of 12 weeks;

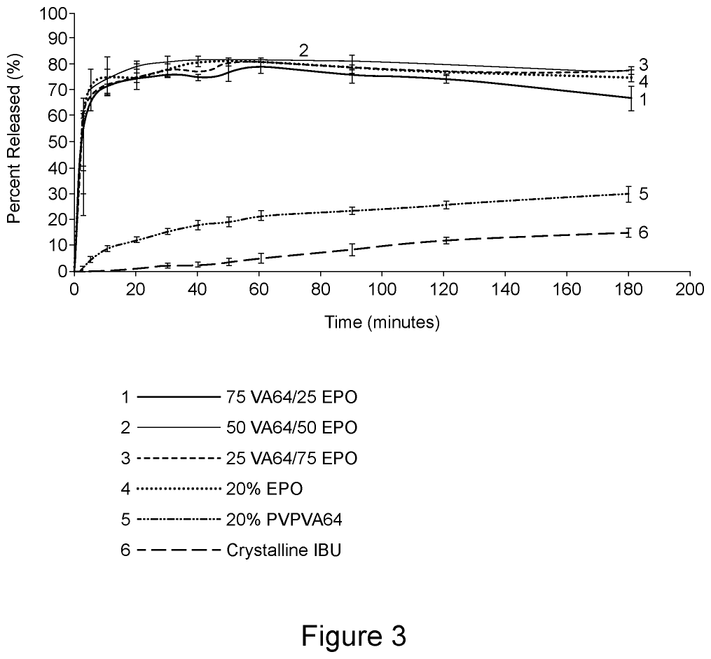

In FIGS. 1-3, the lines in each graph have been given a reference number which corresponds to an ibuprofen-containing composition. The compositions are listed under the graph. In FIG. 1 the reference number 1 corresponds to the results obtained for a composition comprising 20% ibuprofen in EPO, the reference number 2 corresponds to the results obtained for a composition containing 20% ibuprofen in PVPK12, the reference number 3 corresponds to the results obtained for a composition containing 20% ibuprofen in Soluplus, the reference number 4 corresponds to the results obtained for a composition containing 20% ibuprofen in PVP-VA64 and the reference number 5 corresponds to the results obtained for a composition containing ibuprofen only. In FIG. 2, the reference number 1 corresponds to the results obtained for a composition comprising 20% ibuprofen in a 75/25 mixture of PVPK12 and Eudragit, the reference number 2 corresponds to the results obtained for a composition containing 20% ibuprofen in a 50/50 mixture of PVPK12 and Eudragit, the reference number 3 corresponds to the results obtained for a composition containing 20% ibuprofen in a 25/75 mixture of PVPK12 and Eudragit, the reference number 4 corresponds to the results obtained for a composition containing 20% ibuprofen in Eudragit, the reference number 5 corresponds to the results obtained for a composition containing 20% ibuprofen in PVPK12, and the reference number 6 corresponds to the results obtained for a composition containing ibuprofen only. In FIG. 3, the reference number 1 corresponds to the results obtained for a composition comprising 20% ibuprofen in a 75/25 mixture of PVPVA64 and Eudragit, the reference number 2 corresponds to the results obtained for a composition containing 20% ibuprofen in a 50/50 mixture of PVPVA64 and Eudragit, the reference number 3 corresponds to the results obtained for a composition containing 20% ibuprofen in a 25/75 mixture of PVPVA64 and Eudragit, the reference number 4 corresponds to the results obtained for a composition containing 20% ibuprofen in Eudragit, the reference number 5 corresponds to the results obtained for a composition containing 20% ibuprofen in PVPVA64, and the reference number 6 corresponds to the results obtained for a composition containing ibuprofen only.

In FIG. 4, the bars have been given a reference number that corresponds to screw speed. The bar with reference number 1 illustrates the results obtained at 5 rpm. The bar with reference number 2 illustrates the results obtained at 10 rpm. The bar with reference number 3 illustrates the results obtained at 20 rpm. The compositions comprised 20% by weight of ibuprofen. For the compositions which comprised a combination of polymers the relative proportions of each polymer are indicated.

In FIGS. 6 & 7, the period of storage for each composition is shown beside the relevant PXRD pattern. For example, in FIG. 6 the PXRD pattern at the top of the Figure is for a composition stored for a period of 12 weeks. The dotted lines indicated where the diffraction peaks for crystalline ibuprofen would be expected to be appear.

Materials and Methods

Crystalline ibuprofen (MW: 206.2 g/mol) obtained from Shasun (India), Dimethylaminoethyl Methacrylate Copolymer Eudragit® E PO (MW: 147,000 g/mol) obtained from Evonik (Germany), Polyvinylpyrrolidone Kollidon® K12 (MW: 2000-3000 g/mol), Polyvinylpyrrolidone-vinyl acetate copolymers Kollidon® VA64 (MW: 15,000-20,000 g/mol) and polyvinyl caprolactam-polyvinyl acetate-polyethylene glycol graft copolymer Soluplus® (MW: 90,000-140,000 g/mol) obtained from BASF (Germany). All other materials were of analytical grade and used without further treatment.

The combination of two or more polymers provides stability to the amorphous form of the active pharmaceutical ingredient and also inhibits precipitation of the active pharmaceutical agent in the stomach.

Drug distribution between polymers was estimated using infra-red spectrometry (IR).

### Differential Scanning Calorimetry

Thermal behaviour of milled extrudates was evaluated using differential scanning calorimetry DSC 4000 (Perkin-Elmer, Berkshire, UK). 5-10 mg of samples were loaded into aluminium pan sealed by crimping. Samples were analysed using the heat-cool-heat method. Cooling and heating rates were 10° C./min and nitrogen was used as purging gas at a flow rate of 20 ml/min during measurements. A plot of heat flow versus temperature was generated during the measurements and samples were analysed in triplicates.

### Powder X-Ray Diffraction

Solid state characterisations by means of physical state and crystallinity were performed using powder X-ray diffraction. The diffraction patterns of milled extrudates were collected using Miniflex Desktop diffractometer controlled via Standard Measurement software (Rigaku, Japan). Samples were exposed to Cu-Kα radiation under 30 Kv and 15 mA. Samples were packed onto slid sample holder and scanned from 3° to 45° 2θ at scanning speed of 2°/min and step width of 0.02°.

The powder X-ray diffraction patterns for the embodiments shown in FIGS. 6 & 7 were measured using a D2 Phaser X-Ray diffractometer (Bruker, DE). Experimental conditions were selected using the operation software Diffract. Suite Bruker (Bruker, DE) while the data collection was conducted via Diffract Eva (Bruker, DE) software. Powder samples (milled directly before testing where applicable) were manually dispersed into a circular metal sample holder using a stainless-steel spatula and a glass slide with a suitable hand pressure to obtain a levelled surface. Samples were subject to measurements from 3 to 45 2θ angles with step time (0.5 s) and step size (0.02 2θ angles) at 10 mA and 30 kW. The sample holder was set to rotate at 5°/min.

### Infrared Spectroscopy Measurements

Drug-polymer intermolecular interactions were investigated using Fourier-transform infrared (FTIR) spectroscopy in attenuated total reflectance (ATR) mode. Extrudates were analysed using FT/IR 4100 Jesco (JESCO, Japan) fitted with MIRacle ATR accessory (PIKE technologies, USA). Measurements were performed in triplicates over a spectral range of 600 to 4000 cm⁻¹ with 64 scans acquired at a resolution of 4 cm⁻¹. Data were analysed using Spectral Analysis software (JESCO, Japan).

Ibuprofen, Eudragit (EPO) and either PVPK12 or PVPVA64 were extruded using a Rondol 10 mm co-rotating fully intermeshing twin screw extruder (Rondol Technology Ltd, Staffordshire, UK). Barrel temperature was kept at 95° C. during the extrusion process and three screw speeds were used (namely 5, 10, 20 rpm). Full conveying screw geometry was used in all extrusion experiments. Extrudates were stored in glass vials and used for further characterisation. Extrudates based on individual polymers were manufactured at 10 rpm screw speed and 95° C. barrel temperature and used as a standard for comparison. Table 1 below shows the design used to perform the experiments. The extruded compositions comprised 20% ibuprofen, 60% Eudragit E PO and 20% Kolidon VA64.

TABLE 1

| | | | Screw speed | Temperature |
|---|---|---|---|---|
| Run | Polymer I* % | Polymer II** % | (rpm) | (° C.) |
| 1 | 25 | 75 | 5 | 95 |
| 2 | 50 | 50 | 5 | 95 |
| 3 | 75 | 25 | 5 | 95 |
| 4 | 25 | 75 | 10 | 95 |
| 5 | 50 | 50 | 10 | 95 |
| 6 | 75 | 25 | 10 | 95 |
| 7 | 25 | 75 | 20 | 95 |
| 8 | 50 | 50 | 20 | 95 |
| 9 | 75 | 25 | 20 | 95 |

The tensile strength (TS) of produced extrudates was examined using TA XT2i Texture analyser equipped with 50 kg loading cell and controlled via Texture Expert software (Stable micro systems, Surrey, UK). The extrudates were 20 mm long and 2 mm diameter were held between grips set at distance of 10 mm. Test speed was set at 0.5 mm/sec and the final distance to be at 30 mm. All measurements were performed at ambient conditions and samples were tested in triplicates. Force and distance were recorded during measurement using Texture Expert software. The tensile strength of the extrudates was calculated using the following equation: σₘₐₓ=Fₘₐₓ/A where σₘₐₓ is the tensile strength, Fₘₐₓ is the maximum force at break and A is the original area of the extrudates cross-section. The results are shown in FIG. 4. As can be seen the tensile strength of the compositions of the present invention which use a combination of polymers is better than the tensile strength of a composition using only EPO (Eudragit).

### In-Vitro Drug Release Study

The release behaviour of extrudates was investigated using USP TYPE II Apparatus dissolution tester (Copley, UK) operated at 75 rpm and 37.5° C. Milled extrudates were sieved and particles with a size range between 180-300 μm were used for release study. An equivalent amount of 100 mg of ibuprofen was dispersed in the dissolution media. 900 ml of simulated gastric fluid (SGF) without enzymes was used as dissolution media. 2 ml samples were withdrawn at predetermined time points (2.5, 5, 10, 20, 30, 40, 50, 60, 90, 120, and 180 minutes) and filtered into HPLC vials using 0.22 μm modified cellulose acetate syringe filter.

FIG. 1 illustrates the dissolution profiles of the extrudates produced using Eudragit, PVPK12 and PVPVA64. The extrudate based on Eudragit resulted in an improved solubility compared to the extrudates based on PVPK12, PVPVA64 and crystalline ibuprofen. As can be seen from FIGS. 2 & 3, the results of the dissolution testing carried out show that the use of a combination of polymers did not impact significantly on the dissolution profile when compared to a composition using only Eudragit. The use of Eudragit alone resulted in a composition that could not be readily processed.

The concentration of ibuprofen was determined using HPLC system operated at a wavelength of 220 nm using a Diode-Array detector HPLC system Agilent 1220 (Agilent Technology, UK) equipped with an auto-sampler and inline degasser. A reversed-phase Kinetex Evo C18 column with an internal diameter of 4.6 μm, length of 150 mm and pore size of 5 μm was used. Drug release samples were filtered through 0.22 μm cellulose filter and injected using the auto-sampler/injector at a volume of 20 μl. The system was operated at a flow rate of 1 ml/min, a wavelength of 220 nm and the column was kept at a temperature of 25° C. The mobile phase was composed of water (0.1% TFA) and acetonitrile at a ratio of 45:55. The retention time was obtained to be equal to 3.8±0.1 minutes. A stock solution (250 μg/ml) was diluted several times, and the concentration was plotted against the area under the curve (AUC) to construct the calibration curve.

Statistical differences between dissolution data sets were performed using a two-way ANOVA. With respect to tensile strength and drug distribution, one-way ANOVA statistical analysis was performed. The difference was considered to be significant at p<0.05. Post-hoc Tukey's HSD test was performed for data sets that showed significant statistical differences. Statistical analysis was performed using Graphpad Prism 7.0 (GraphPad Software, USA).

Without being bound by any theory, it is believed that despite being problematic for low Tg polymers, the ibuprofen plasticising effect is beneficial and makes possible the processing of high Tg polymers such as PVPK12 and PVPVA64 at temperatures below their Tg.

Extrusion of 20% (wt %) drug-loaded formulations using either PVPK12 or PVPVA64 only was performed at 95° C. and 10 rpm screw speed. Clear strands were produced and used as a standard for comparison.

Solid state characterisation using PXRD and DSC confirmed a complete formation of the glass solutions manifested by the halo-like PXRD pattern and a single glass transition temperature The Tg of PVPK12, PVPVA64 and Soluplus were reduced to 58±2.1° C., 64±1.9° C. and 36±1.4° C., respectively. This can be explained by the ability of ibuprofen to plasticize the polymers as mentioned previously.

Significant dissolution improvement for all formulations was observed when compared with crystalline ibuprofen. This can be attributed to the solubility advantage of the amorphous form, and the presence of Eudragit in all the formulations which showed an excellent inhibition of ibuprofen precipitation from supersaturated solutions.

The Eudragit concentration in the dissolution media can explain the differences in the percentage of ibuprofen released as the ability of Eudragit to inhibit ibuprofen precipitation is concentration-dependent.

The alteration of polymer ratio in the formulation did not solely contribute to the change in the dissolution behaviour of the formulations. A change in screw speed also caused a change in dissolution behaviour as illustrated previously with formulations based on PVPVA64/EPO.

When processed at low screw speed, formulations revealed the higher extent of precipitation inhibition, whereas those produced at higher screw speed showed the lowest extent of precipitation inhibition.

Changing the screw speed has an effect on the residence time inside the extruder which in turn may affect the extent of polymer softening. Sufficient polymer softening is crucial for drug dispersion, as insufficient softening may impede drug diffusion into polymer matrix. In addition to the residence time, the mechanical energy input induced by screw agitation would increase as screw speed increased. Together, these factors could affect drug distribution between both polymers and hence affect the extrudates mechanical and dissolution behaviour.

The use of a second polymer improves the mechanical properties of ibuprofen extrudates based on EPO. For the extrudates that contain different amounts of the polymers, the tensile strength was improved regardless of the amount incorporated, when compared with EPO based extrudates. The mechanical properties of the formulations can be tailored to suit a particular requirement by using a different screw speed. This is believed to be as a result of the distribution of ibuprofen between both polymers. FTIR data shows that as the screw speed increases, drug distribution into EPO decreases. This was perceived as an improvement in the mechanical properties of the extrudates as a higher percent of ibuprofen is distributed to the other polymer (which have a higher Tg) rather than EPO. Table 2 below illustrates the amounts of ibuprofen in EPO and PVPVA64 at different temperatures and screw speeds.

TABLE 2

| Polymer Ratio | Screw Speed | Percent in EPO | Percent in PVPVA64 |
|---|---|---|---|
| 75 PVPVA64/ | 5 | 37% | 63% |
| 25 EPO | 10 | 32% | 68% |
| | 15 | 23% | 77% |
| 50 PVPVA64/ | 5 | 49% | 51% |
| 50 EPO | 10 | 42% | 58% |
| | 15 | 30% | 70% |
| 25 PVPVA64/ | 5 | 77% | 23% |
| 75 EPO | 10 | 66% | 34% |
| | 15 | 47% | 53% |

The dissolution studies of the formulations of the present invention indicate that precipitation inhibition was not solely controlled by the concentration of EPO in the dissolution media. The dissolution rate of PVP polymers was also seen to affect the extent of ibuprofen precipitation concomitantly.

FIG. 5 illustrates the dissolution profile for compositions comprising 40% by weight of ibuprofen, 55% by weight of Eudragit E PO and 5% by weight of Kolidon VA64 (Example 1) and 40% by weight of ibuprofen, 55% by weight of Eudragit E PO and 5% by weight of Kolidon VA64 (Examples 2 & 3). Example 3 had 1% by weight added to the extrudate prior to the dissolution test being carried out. The dissolution profile for each composition is set out in Table 3 below.

TABLE 3

| Time/mins | Example 1 | Example 2 | Example 3 |
|---|---|---|---|
| 0.0 | 0.0 | 0.0 | 0.0 |
| 5.0 | | 17.2 | 18.9 |
| 10.0 | 50.0 | 46.9 | 47.9 |
| 15.0 | | 55.4 | 55.7 |
| 20.0 | 59.6 | 58.0 | 56.6 |
| 30.0 | 58.4 | 59.2 | 57.3 |
| 45.0 | 58.6 | 58.8 | 56.9 |
| 60.0 | 57.8 | 59.0 | 58.3 |

It was found that for extrudate compositions comprising 40% by weight of ibuprofen that changes to the screw speed of the extruder did not impact on the dissolution profile of the composition.

The data obtained with regard to the tensile strength and dissolution behaviour illustrates the importance of the polymer combination. By incorporating a second polymer the amount of EPO could be reduced without affecting the drug release behaviour.

An advantage of the present invention is that there is provided a formulation containing ibuprofen in an amorphous form which further comprises two polymers with different physicochemical properties that maximise the mechanical properties and the dissolution behaviour of extruded formulations.

Further modifications and developments can be made without departing from the scope of the invention described herein.

1. A solidified melt extrudate comprising an active pharmaceutical ingredient in an amorphous form and a combination of at least two polymers.

2. The solidified melt extrudate as claimed in claim 1, wherein the active pharmaceutical ingredient is a nonsteroidal anti-inflammatory drug (NSAID) or paracetamol.

3. The solidified melt extrudate as claimed in claim 2, wherein the NSAID is selected from the group consisting of ibuprofen, flurbiprofen, ketoprofen, diclofenac, naproxen, aspirin, indomethacin, and meloxicam.

4. (canceled)

5. The solidified melt extrudate as claimed in claim 1, wherein the extrudate comprises from 10% by weight to 50% by weight of the active pharmaceutical ingredient.

6. The solidified melt extrudate as claimed in claim 5, wherein the extrudate comprises from 20% by weight to 40% by weight of the active pharmaceutical ingredient.

7. The solidified melt extrudate as claimed in claim 1, wherein the first polymer of the combination of at least two polymers is selected from polymers having a glass transition temperature of at least 30° C.

8. The solidified melt extrudate as claimed in claim 7, wherein the first polymer has a glass transition temperature of between 40° C. and 50° C.

9. (canceled)

10. (canceled)

11. The solidified melt extrudate as claimed in claim 10, wherein the first polymer is dimethylaminoethyl methacrylate co-polymer.

12. (canceled)

13. (canceled)

14. The solidified melt extrudate as claimed in claim 1, wherein the second polymer of the combination of two polymers is selected from polymers having a glass transition temperature of at least 80° C.

15. The solidified melt extrudate as claimed in claim 14, wherein the polymer has a glass transition temperature of between 90° C. and 110° C.

16. (canceled)

17. The solidified melt extrudate as claimed in claim 14, wherein the second polymer of the combination of two polymers is selected from the group consisting of polyvinylpyrrolidone, polyvinylpyrrolidone-vinyl acetate co-polymer and polyvinyl caprolactam-polyvinyl acetate-polyethylene glycol graft copolymer.

18. The solidified melt extrudate as claimed in claim 17, wherein the second polymer is polyvinylpyrrolidone K12 or polyvinylpyrrolidone-vinyl acetate co-polymer (PVPVA64).

19. (canceled)

20. (canceled)

21. The solidified melt extrudate as claimed in claim 1, wherein the combination of the first and second polymers is dimethylaminoethyl methacrylate co-polymer and polyvinylpyrrolidone-vinyl acetate co-polymer (PVPVA64).

22. (canceled)

23. The solidified melt extrudate as claimed in claim 1, wherein the ratio of the first polymer and the second polymer of the combination of two polymers is selected to be from 10:1 to 1:10.

24. (canceled)

25. (canceled)

26. (canceled)

27. (canceled)

28. (canceled)

29. (canceled)

30. (canceled)

31. (canceled)

32. (canceled)

33. (canceled)

34. The solidified melt extrudate as claimed in claim 1, wherein the combination of polymers is soluble at a pH of about 1 to about 5.

35. The solidified melt extrudate as claimed in claim 34, wherein the combination of polymers is soluble at a pH of about 2 to about 4.

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. The solidified melt extrudate as claimed in claim 1, wherein the extrudate comprises an NSAID or paracetamol in an amorphous form and a combination of a first polymer having a glass transition temperature of between 40° C. and 50° C. and a second polymer having a glass transition temperature of between 90° C. and 110° C., and wherein the extrudate comprises from 30%-45% by weight NSAID or paracetamol.

48. The solidified melt extrudate as claimed in claim 47, wherein when the second polymer of the combination of two polymers is selected to be a homopolymer, the second polymer has an average molecular weight of 1,000 to 5,000 Daltons and when the second polymer of the combination of two polymer is selected to be a co-polymer, the second polymer has an average molecular weight of from 45,000 to 70,000 Daltons.

49. The solidified melt extrudate as claimed in claim 47, wherein the weight ratio of the first polymer, and the second polymer is 5:1 to 15:1.

50. The solidified melt extrudate as claimed in claim 47, wherein the weight ratio of the NSAID, the first polymer, and the second polymer is 5:8:1 to 10:15:1.

51. The solidified melt extrudate as claimed in claim 47, wherein the solidified melt extrudate comprises 30%-45% by weight of the NSAID in an amorphous form and a combination of 45%-65% by weight of the first polymer and 1%-10% by weight of the second polymer.

52. The solidified melt extrudate as claimed in claim 51, where the NSAID is ibuprofen.

53. The solidified melt extrudate as claimed in claim 1, wherein the solidified melt extrudate comprises ibuprofen in an amorphous form and a combination of dimethylaminoethyl methacrylate co-polymer and polyvinylpyrrolidone-vinyl acetate co-polymer (PVPVA64), and wherein the extrudate can comprise from 30%-45% by weight NSAID.

54-62. (canceled)
